(19) United States
(12) Patent Application Publication
(10) Pub. No.: US 2003/0082509 A1
(43) Pub. Date: May 1, 2003

(54) Compositions for freezing dog sperm and method of freezing the dog sperm utilizing the compositions and artificial insemination method employing the frozen dog sperm

(76) Inventor: Son Hwa Sub, Seongnam-city (KR)

Correspondence Address: PENINSULA IP GROUP, A Professional Law Corporation, Suite 101, 2290 North First Street, San Jose, CA, 95131, US

(21) Appl. No.: 10/040,290

(22) Filed: Oct. 25, 2001

(51) Int. Cl. A01N 1/02; C12N 15/00

(52) U.S. Cl. 435/2; 800/21

(57) ABSTRACT

Disclosed herein is a composition and method for freezing dog sperm. The composition is based upon 100 ml of distilled water, 0.25 to 4.5 g of sodium citrate, 0.45 to 4.0 g of dextrose, 0.01 to 0.3 g of penicillin, 50,000 to 500,000 IU of streptomycin. 0.25 to 0.45 g of catalase, 0.045 to 0.25 g of yolk, 1.5 to 15 ml of glycerol and 0.02 to 0.1 ug of dog serum. The method for freezing dog sperm includes the steps of (a) collecting male dog sperm by a massage method or electrical shock, (b) centrifuging the collected sperm to separate and remove the upper fluid leaving only the lower sperm, (c) mixing equal volumes of the lower sperm and the composition and primarily refrigerating the mixture, (d) further adding the composition to the mixture obtained in the step (c) to reduce the volumetric ratio of sperm to the composition to 1:2, and secondarily refrigerating the resulting mixture, and (e) adding liquid nitrogen to the mixture obtained in the step (d) and freezing the mixture.

### BACKGROUND OF THE INVENTION

1. Field of the Invention

The present invention relates to compositions for freezing dog sperm, a method of freezing the dog sperm utilizing the compositions and an artificial insemination method employing the frozen dog sperm. More particularly, the present invention relates to compositions for freezing dog sperm in which the number of spermatozoa living in the sperm and the shape and viability thereof are retained when the sperm is thawed within a predetermined time after it is frozen, a method of freezing the dog sperm utilizing the compositions, and an artificial insemination method involving thawing the frozen dog sperm at a suitable place and time and inserting the same into the vagina of the female dog.

2. Description of the Related Art

According to recent industrial development, genetic engineering has become an important field of study. Genetic engineering is necessary for the preservation and development of good breeds and the prolongation of the human life span, and has advanced to a considerably high level, beyond the fundamental stage.

In the case of breeding livestock or pets, artificial insemination using liquid sperm allows fertilization of several female animals by mixing the sperm collected from a male animal with a diluent fluid to increase the volume of fluid available for injection. Thus, in performing the artificial insemination, superior or pure sperm is collected and several female animals can be simultaneously fertilized, thereby producing superior breeds and preserving pure breeds.

In order to achieve successful artificial insemination having several advantages described above, the development of proper diluent fluids becomes an important issue. Since research into diluent fluids started several tens of years ago, diluent fluids applicable to pigs, horses and cattle have been developed and put into practice. However, the development of diluent fluids applicable to dog sperm has not yet matured.

A method of preserving animal sperm for a long time without loss of freshness would allow superior sperm to be used to fertilize female animals as necessary, which would be a great contribution to the development of livestock and the pet industry.

A freezing method is most suitable for preserving sperm for a long time. However, a freezing method for dog sperm is particularly underdeveloped. Although not yet clearly defined, the specific reason for the underdevelopment is presumably that reagents for freezing male dog sperm spermatozoa of which have peculiarity, and freezing methods using the reagents are not yet sufficiently developed.

### SUMMARY OF THE INVENTION

It is a first object of the present invention to provide compositions for freezing dog sperm, by which dog spem1 having peculiar spermatozoa can be frozen for storage.

It is a second object of the present invention to provide a method of freezing sperm collected from a male dog using the compositions, by which the sperm can be stored for along time and can be conveniently carried, and by which the frozen dog sperm can be thawed at an appropriate time for artificial insemination.

It is a third object of the present invention to provide an artificial insemination method using the frozen sperm based on the freezing method, by which superior animals Carl be bred, and which can be put into widespread distribution.

To accomplish the first object, there is provided a composition for freezing dog sperm comprising, based on 100 ml of distilled water, 0.25 to 4.5 g of sodium citrate, 0.45 to 4.0 g of dextrose, 0.01 to 0.3 9 of penicillin, 50,000 to 500,000 IU of streptomycin, 0.25 to 0.45 g of catalase, 0.045 to 0.25 g of yolk, 1.5 to 15 ml of glycerol and 0.02 to 0.1 g of dog serum.

To accomplish the second object, there is provided method for freezing dog sperm using the composition, including the steps of (a) collecting male dog sperm by a massage method or electrical shock, (b) centrifuging we collected sperm to separate and remove the upper fluid leaving only the lower sperm, (c) mixing equal volumes of the lower sperm and a composition and, primarily refrigerating the mixture, (d) further adding the composition to the mixture obtained in the step (c) to reduce the volumetric ratio of sperm to the composition to 1:2, and secondarily refrigerating the resulting mixture, and (e) adding liquid nitrogen to the mixture obtained in the step (d) and freezing the mixture.

To accomplish the second object, there is provided a method for artificial insemination of dogs including the steps of (a) examining an appropriate ovulatory phase of a female dog for improvement of a fertility rate, (b) adding the frozen SpeTn1 prepared by the method of claim 2 to a mixture of 0.3 to 1.5 wt % sodium The present invention will now be described in more detail.

Compositions for freezing dog sperm will first be described.

The compositions for freezing dog sperm according to the present invention are prepared by homogeneously mixing into 100 ml of distilled water, 0.25 to 4.5 9 of sodium nitrate for regulating the buffering function, acidity (pH) and osmotic pressure of spermatozoa, 0.45 to 4.0 g of dextrose as a metabolism and energy source, 0.01 to 0.3 g of penicillin as an antibiotic for preventing il1fection, 50,000 to 500,000 IU of streptomycin, 0.25 to 0.4.5 g of catalase, 0.045 to 0.25 g of yolk, 1.5 to 15 ml of glycerol and 0.02 to 0.1 ug of dog serum. The thus prepared composition is frozen in units of about 2 ml to be thawed whenever it is to be used.

The composition according to the present invention stores sperm for a long time while maintaining the viability and motility of spermatozoa contained in the frozen sperm, and contains dog serum exhibiting normal fertility and delivery rates after thawing.

Next, a method for freezing dog sperm using the composition will be described.

It is necessary for the freezing of dog sperm to first collect the same. As the sperm collection method, known methods in the art can be used without restriction, preferably a massaging method or electrical shock. The massaging method that is most widely used will now be described briefly.

Since spermatozoa die quickly when mixed with urine, it is preferred for a male dog to urinate before sperm is collected from the male dog. Then, the genital organ of the male dog is massaged using a test tube having an artificial vagina inserted therein to make the male dog ejaculate into the test tube. To catalyze the collection of sperm by this method, a female dog in heat is let to hover around the male dog. Otherwise, frozen cotton or a sponge containing a vaginal secretion of a female dog in heat is thawed and allowed to be smelled by the male dog when collecting sperm.

In the case of collecting sperm using the massaging method, the sperm is divided into three samples collected at different tubes. Here, the sample of the sperm containing spermatozoa is the second one collected. Each sample is collected at a time interval of about 10 to 20 seconds. However, in some cases, the interval between first and second samples is too short to be discernible. In the present invention, the second sample is preferably used. However, in the case when it is not possible to discern the first sample from tile second sample, three samples are all collected in one test tube to be used.

Then, the thus collected sperm is separated by means of a centrifuge, and the upper fluid is discarded with only the lower sedimentary sperm left. Centrifuging is performed at about 500 to 1100 RPM/minute for 6 minutes.

Equal volumes of the lower sedimentary sperm and the composition prepared as described above are mixed at room temperature and then the mixture is primarily refrigerated. Here, the refrigerating time is about 10 to 20 minutes and the refrigerating temperature is about 5″ C. A mixture of sperm and the composition in a volumetric ratio of 1:2 is further mixed with the primarily refrigerated mixture at room temperature, and the secondarily refrigerated at the same temperature and for the same time. Through the primary and secondary refrigerating steps, the sperm and the composition are homogeneously mixed.

Next, a predetermined amount of the secondarily refrigerated mixt1lre is taken by means of a dropper and 2-3 drops are dropped to the surface of dry ice. Since the surface temperature of dry ice is about −79 C, the mixture is frozen to an extent. The frozen mixture is separated from dry ice and placed into a container having liquid nitrogen at about −196 C to then be heffi1etically sealed, thereby completing the process of freezing dog sperm. This method is called a “pellet method” since the frozen mixture in. the liquid nitrogen is pellet-shaped.

In addition to the above-described pellet method, the dog sperm can be frozen by a method using a straw, which will now be described briefly.

The secondarily refrigerated mixture is subdivided into about 0.5 ml samples and put into straws. Every 20 to 30 straws are grouped and immersed in a container containing liquid nitrogen. When the straws are immersed into the container for freezing, they are gradually immersed, rather than immersed to the bottom of the container at once. First, they are immersed as deep as the upper portion of the container to then be left there for a predetermined time, then as deep as the middle of the container to then be left there for a predetermined time, and then as deep as the bottom of the container.

The thus frozen sperm can be carried and stored for a long time, and can be used for artificial insemination after being thawed as needed.

Next, an artificial insemination method using the frozen sperm will be described.

First, in order to perform artificial insemination, a test is performed to determine whether the female dog is in an ovulatory phase. The ovulatory phase test is performed by known methods in the art to which the present invention pertains, such as a vaginal cell test or a hormone test.

Next, the frozen sperm is thawed by adding the frozen sperm to a mixt1lre of 0.3 to 1.5 wt % sodium chloride solution and a small amount of dog sperm heating, the mixture heated to a temperature of 30 to 40 C. which is advantageous for maintaining the viability and shape of spermatozoa. Also, although the mixture may be directly heated, since it is quite difficult to appropriately control the temperature it is preferred that an airtight container containing the mixture of sodium chloride solution and dog sperm is submerged in hot water at about 50 C and heated.

Prior to using the thus thawed sperm in artificial insemination, the number, shape and mobility of living spermatozoa are examined through a microscope. Successful artificial insemination depends on the number of spermatozoa moving forward. Spermatozoa are classified into four types based on their mobility.

Class 0 means me case: where spermatozoa move their tales without forward movement, class 1 means the case where less than 20% of spermatozoa move forward, class 2 means the case where 20 to 50% of spern1atozoa move forward, class 3 means the case where 50 to 80% of spermatozoa move forward, and class 4 means the case where more than 80% of spermatozoa move forward. When the thawed sperm is placed on a slide and observed through a microscope, only the sperm of mobility class 2 or higher can be used for artificial insemination.

Then, the thawed sperm is injected into the vagina of the female dog detern'1ined to be in the ovulatory phase and the clitoris of the female: dog is massaged. to complete artificial insemination.

In addition to the above-described artificial insemination method, the thawed sperm may be injected into the uterus of the female dog in a surgical manner. In other words, the lower part of the navel is cut about 2 cm to raise the uterus and the sperm of the male dog is directly injected into the uterus of the female dog by means of an injection syringe. Then, the peritoneum abdominal muscle and skin of the female dog are sewed in order.

### BRIEF DESCRIPTION OF THE DRAWING

There are no drawings.

### DETAILED DESCRIPTION OF THE INVENTION

This invention is further illustrated by the: following examples, which are not to be construed in any way as imposing limitations upon the scope of the invention.

### EXAMPLE 1

Preparation of Composition for Freezing Dog Sperm

A mixture having the following composition was homogeneously mixed using a mixer to prepare the composition for freezing dog sperm based on 100 ml of distilled water.

Sodium citrare 0.35 g

Dextrose 2.5 g

Penicillin 0.02 g

Streptomycin 100,000 IU

Catalase 0.35 g

Yolk 0.05 g

Glycerol 2 ml

Dog serum 0.05 ug

Freezing Method of Dog Sperm

Sperm was collected from 6 year old shepherd dog weighing 43 kg by electrical shock. The amount of collected sperm was 6.2 ml. Next, the collected sperm was separated by means of a centrifuge to remove the upper fluid with only the lower sediment left over. Centrifuging was performed at 500 to 1100 RPM/min for 6 minutes.

Equal volumes of the thus separate lower sperm and the composition prepared in the above-described manner were mixed at room temperature and then primarily refrigerated. Here, the refrigerating time was 10 to 20 minutes and the refrigerating temperature was about 5° C. The composition was further added to the primarily refrigerated mixture to then be mixed such that the mixture ratio of the sperm to the composition by volume became 1:2. This resulting mixture was then secondarily refrigerated at the same temperature and for the same time.

Next, a predetermined amount of the secondarily refrigerated mixture was taken by means of a dropper and 2-3 drops were dropped onto the surface of dry ice. Since the surface temperature of dry ice is about −79° C., the mixture was frozen to an extent. The frozen mixture was separated from the dry ice and placed into a container having liquid nitrogen of about −196° C., to then be hermetically sealed, thereby completing the freezing process of dog sperm.

Artificial Insemination Using Frozen Sperm

Three 3-year old female shepherd dogs weighing 30 kg were used for artificial insemination. It was examined by a vaginal cell test whether or not the female dog were in an ovulatory phase.

Now, it is necessary to thaw the sperm frozen in the above-described manner, Thawing was performed such that the frozen sperm was added to a mixture of 0.3 to 1.5 wt % sodium chloride solution and a small amount of dog sperm, the mixture was heated to a temperature of 38″C and was then let to sit for 5 hours.

Then, prior to using the thawed sperm for artificial insemination, the mobility of living spermatozoa was examined by placing the thawed sperm on a slide and observing the same through a microscope. Successful artificial insemination depends on the number of spermatozoa moving forward. The percentage of spermatozoa moving forward was about 85%, which is suitable for artificial insemirultiol1. Next, the thawed sperm was divided into three portions and then injected into the vaginas of female dogs determined to be in an ovulatory phase. Then, the clitoris of each of tile female dogs was massaged for 5 minutes, to complete artificial insemination.

A pregnancy test was done 1 month after completion of artificial insemination, arid all three female dogs turned out to be pregnant. Later, the three female dogs delivered 4, 6 and 9 healthy puppies, respectively.

### EXAMPLE 2

In this example, tile composition for freezing dog spem1 was prepared in the same manner as in Example I, with the exception of 0.02 ug of dog serum being used.

Subsequently, the freezing of dog sperm and artificial insemination were performed using the composition, by the same process as described in example 1.

In this embodiment, a 5-year old male shepherd dog weighing 40 kg and three, 3 year old female shepherd dogs weighing 25 kg, 30 kg and 28 kg were used.

The mobility test result of thawed sperm showed that the percentage of spermatozoa moving forward was 60%, which was a suitable condition for artificial insemination.

A pregnancy test was done 1 month after completion of artificial insemination, and two female dogs turned out to be pregnant. Later, the two female dogs delivered 5 and 6 healthy puppies, respectively.

### COMPARATIVE EXAMPLE 1

In this comparative example1 the same composition for freezing dog sperm as in Example 1 was prepared and the dog sperm was frozen in the same manner as in Example, with the exception of the volumetric ratio of the composition to the dog sperm being 1:1. The dog sperm was collected in the same manner as in Example 1 using the same male dog.

The frozen sperm was thawed in the same manner as in Example 1 and the mobility of spermatozoa in the thawed sperm was tested. The test result showed that the percentage of spermatozoa moving forward was less than 20%, which is not suitable for artificial insemination.

### COMPARATIVE EXAMPLE 2

In this comparative example, the same composition for freezing dog sperm was prepared in the same manner as in Example 1, with the exception of dog serum not being contained in the composition. The freezing and thawing methods were the same as those in Example 1, and the dog sperm was collected in the same manner as in Example 1 using the same male dog.

The mobility test of spermatozoa frozen and thawed according to this comparative example showed that the spermatozoa move with their tales only without forward movement, which is not suitable for artificial insemination.

As described above, the present invention provides a method for freezing dog sperm, which is difficult to be frozen for storage in view of peculiarity of spermatozoa of the dog sperm, and compositions for use therein. Therefore, the dog sperm can be stored in a frozen state, thereby preserving the dog of good strain. Also, the frozen male dog sperm can be used for artificial insemination at an appropriate time and place, thereby enabling an increase in the birth rate.

What is claimed is:

1. A composition for freezing dog sperm comprising, based on 100 ml of distilled water, 0.25 to 4.5 9 of sodium citrate, 0.45 to 4.0 9 of dextrose, 0.01 to 0.3 9 of penicillin. 50,000 to 500,000 IU of streptomycin. 0.25 to 0.45 9 of catalase, 0.045 to 0.25 9 of yolk, 1.5 to 15 ml of glycerol and 0.02 to 0.1 ug of dog serum.

2. A method for freezing dog sperm using the composition claimed in claim 1, comprising the steps of:
(a) collecting male dog sperm by a massage method or electrical shock;
(b) centrifuging the collected sperm to separate and remove the upper fluid leaving only the lower sperm;
(c) mixing equal volumes of the lower sperm and a composition and primarily refrigerating the mixture;
(d) further adding the composition to the mixture obtained in the step (c) to reduce the volumetric ratio of sperm to the composition to 1:2, and secondarily refrigerating tl1e resulting mixture; and
(e) adding liquid nitrogen to the mixture obtained in the step (d) and freezing me mixture.

3. A method for artificial insemination of dogs comprising the steps of:
(a) examining an appropriate ovulatory phase of a female dog for improvement of a fertility rate;
(b) adding the frozen sperm prepared by the method of claim 2 to a mixture of a 0.3 to 1.5 wt % sodium chloride solution and dog serum, heated to a temperature of 30 to 40Q C and thawing the same;
(C) testing the mobility of spermatozoa in the thawed sperm through a microscope; and
(d) injecting the thawed sperm into the vagina of a female dog and massaging the clitoris of the female dog.
